(19) United States
(12) Patent Application Publication
(10) Pub. No.: US 2004/0067871 A1
(43) Pub. Date: Apr. 8, 2004

(54) Genetic determinants for dermal inflammation and immunity

(76) Inventors: William Osmond Charles Cookson, Oxford (GB); John Harper, London (GB); Miriam Fleur Moffatt, Oxford (GB); Goncalo Rocha Abecasis, Oxford (GB)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/381,932

(22) PCT Filed: Sep. 28, 2001
(86) PCT No.: PCT/GB01/04338

(30) Foreign Application Priority Data
Sep. 29, 2000 (GB) 0023947.5

(51) Int. Cl. A61K 31/00; C12Q 1/68

(52) U.S. Cl. 514/1; 435/6

(57) ABSTRACT

The invention relates to the localisation of genetic factors associated with dermal inflammation and immunity to particular regions of the human genome. In particular, there are described methods of determining the genetic susceptibility of an individual to disease conditions involving dermal inflammation and immunity, especially but not exclusively atopic dermatitis, methods for the identification of polymorphic alleles which confer susceptibility to conditions involving dermal inflammation and immunity and methods for the identification of a human genes which contribute to or are responsible for the manifestation of disease conditions involving dermal inflammation and immunity.

The invention is concerned with the determination of genetic factors associated with dermal inflammation and immunity. In particular, although not exclusively, the invention is concerned with determining the genetic susceptibility of an individual to disease conditions involving dermal inflammation and immunity, especially but not exclusively atopic dermatitis. The invention is also concerned with methods for the identification of polymorphic alleles which confer susceptibility to conditions involving dermal inflammation and immunity and to methods for the identification of a human genes which contribute to or are responsible for the manifestation of disease conditions involving dermal inflammation and immunity.

Atopic dermatitis (AD, eczema) affects 10-20% of children in westernised societies and shows a strong shared familial aggregation with asthma. AD commonly begins in infancy and early childhood, and is typified by itchy, inflamed skin. Infants with AD are prone to weeping inflammatory patches and crusted plaques on the face, neck, extensor surfaces, and groin. Children and young adults tend to have dermatitis of flexural skin, particularly in the antecubital and popliteal fossae. Atopy is a prominent feature of AD. The atopic state is characterised by Immunoglobulin E (IgE) responses to common allergens such as house dust mite proteins. Eighty % of cases of AD have elevations of the total serum IgE concentration (Juhlin et al., 1969), and asthma is present in 60% of children with severe AD (Cox et al., 1998). Consequently, atopic mechanisms dominate current understanding of the pathogenesis of AD (Bos, 2000).

Twin studies of AD have shown concordance rates of 0.72-0.86 in monozygotic and 0.21-0.23 in dizygotic twin pairs, suggesting the presence of strong genetic factors in the development of disease (Shultz Larsen, 1993; Williams, 1994). The present inventors have therefore investigated the genetic predisposition to AD by carrying out a genome-wide screen for linkage to AD, asthma, and the total serum IgE concentration. The genome-wide screen of nuclear families ascertained through a child with severe AD identified evidence for linkage (p<0.001) to AD or to AD and asthma combined on chromosomes 1q21, 17q25, and 20p. These three loci correspond very closely with known psoriasis susceptibility loci, and the 1q21 locus contains a cluster of genes regulating epidermal differentiation. The conservative probability of the AD and psoriasis loci coinciding by chance was estimated by simulation to be<2.8×10⁻⁵. The results suggest the presence of polymorphic loci with general effects on dermal inflammation and immunity and indicate that AD is influenced by genes modulating dermal responses, in addition to those promoting the general atopic state.

The linkage regions identified by the inventors may be used to locate susceptibility gene(s) which have a general effect on dermal inflammation and immunity and may further be scanned for the presence of polymorphisms which may be associated with susceptibility to disease conditions involving dermal inflammation and immunity.

The term “disease conditions involving dermal inflammation and immunity” encompasses, but is not limited to, atopic dermatitis, psoriasis and allergic contact dermatitis.

Accordingly, in a first aspect the invention comprises the use of a region of human chromosome 1q21 including polymorphic marker D1S498 for identifying a polymorphic allele which is associated with susceptibility to disease conditions involving dermal inflammation or immunity.

The invention also comprises use of a region of human chromosome 17q25 including polymorphic marker D17S784 for identifying a polymorphic allele which is associated with susceptibility to disease conditions involving dermal inflammation or immunity.

The invention still further comprises use of a region of human chromosome 20p including polymorphic marker D20S115 for identifying a polymorphic allele which is associated with susceptibility to disease conditions involving dermal inflammation or immunity.

The present inventors have identified three regions of the human genome which are associated with a general susceptibility to dermal inflammation and immunity, showing linkage to both atopic dermatitis and psoriasis. In this first aspect, the invention provides for use of these regions of the genome (referred to as linkage regions) in the identification of polymorphic variants associated with general susceptibility to disease conditions involving dermal inflammation and immunity. In a further embodiment the invention also contemplates use of the linkage regions identified herein in the identification of polymorphic variants associated with susceptibility to particular disease conditions involving dermal inflammation and immunity, a preferred example being atopic dermatitis.

The first linkage region identified by the inventors is on human chromosome 1q21 and includes marker D1S498. This first linkage region may be further defined with reference to markers which flank the marker at the peak of linkage, thus in a preferred embodiment the invention provides for use of a linkage region of human chromosome 1q21 disposed between markers D1S2726 and D1S2878 which flank D1S498 at positions 144 cM and 178.4 cM from the top of the linkage group respectively, or a sub-region of this region. The sub-region may or may not include the polymorphic marker D1S498.

The second linkage region is on human chromosome 17q25 and includes marker D17S784. This second linkage region may be further defined with reference to markers which flank the marker at the peak of linkage, thus in a preferred embodiment the invention provides for use of a linkage region of human chromosome 17q25 disposed between marker D17S785 (at position 103.8 cM) and the telomere, or a sub-region of this region. The sub-region may or may not include the polymorphic marker D18S784.

The third linkage region is on human chromosome 20p and includes marker D20S115. This third linkage region may be further defined with reference to markers which flank the marker at the peak of linkage, thus in a preferred embodiment the invention provides for use of a linkage region of human chromosome 20p disposed between markers D20S117 and D20S112 which flank D20S115 at positions 2.8 cM and 39.7 cM from the top of the linkage group respectively, or a sub-region of this region. The sub-region may or may not include the polymorphic marker D20S115.

Markers D1S2726, D1S2878, D17S785, D20S117 and D20S112 are all publicly available and are shown on the Marshfield map (Broman, K. W. et al., 1998).

The linkage regions identified herein may be scanned for the presence of polymorphic alleles which may be associated with dermal inflammation and immunity using techniques well known in the art of human genetics. A preferred approach would be to scan the linkage regions in a number of individuals, including normal or healthy individuals and individuals afflicted with conditions involving dermal inflammation and immunity, such as atopic dermatitis (patients), and thus identify any polymorphic variation within the linkage region. The term “polymorphic variation” includes, but is not necessarily limited to, single nucleotide polymorphisms and other ‘simple’ polymorphisms such as short deletions or insertions and multinucleotide changes and also restriction fragment length polymorphisms.

For any polymorphic variants identified in the linkage region, association with dermal inflammation or immunity in general or with a particular disease condition, for example atopic dermatitis, may be evaluated using techniques known in the field of human genetics, such as family-based association studies.

The identification of polymorphic alleles associated with dermal inflammation and immunity is of importance in the development of diagnostic tests, for example genetic screens to identify individuals susceptible to development of conditions associated with dermal inflammation and immunity.

The actual step of scanning the linkage region for the presence of polymorphic variants may be accomplished using any of the techniques known in the art (see review by Schafer and Hawkins, Nature Biotechnology, Vol 16, pp33-39 (1998)). Preferred techniques are listed below, however this list is not to be construed in any way as limiting the scope of the invention:

(a) DNA sequencing: Heterozygous changes appear as two bases at a single position in the sequence. Homozygous variants are found by comparison to a control (i.e. wild-type) sequence.

(b) Heteroduplex analysis: this technique is based on the fact that heteroduplexes exhibit a reduced mobility in non-denaturing polyacrylamide gels compared to homoduplexes. The region to be tested (advantageously around 200 bp) is amplified, denatured and re-natured to itself or control ‘wild-type’ DNA and the duplexes resolved on a non-denaturing gel. The same region of DNA is compared between individuals and differential mobilities indicate sequence differences.

(c) Single-strand conformation polymorphism analysis (SSCP or SSCA): single stranded DNA folds up to form complex structures that are stabilized by weak intramolecular bonds. The electrophoretic mobilities of these structures on non-denaturing polyacrylamide gels is dependent upon chain length and conformation. Typically, PCR amplification products from the region to be tested are heat denatured and rapidly cooled to impede reassociation of complementary strands. The products are then resolved on a non-denaturing gel. The same region of DNA is compared between individuals and differential mobilities indicate sequence differences that exist between the individuals in this region.

(d) Chemical cleavage of mismatches (CCM): a radiolabelled probe is hybridised to the test DNA and mismatches detected by a series of chemical reactions that cleave one strand of the DNA at the site of the mismatch. This sensitive method can be applied to kilobase-length fragments.

(e) Enzymatic cleavage of mismatches: technique similar to CCM, except that the cleavage is performed using an enzyme (e.g. T4 endonuclease VII).

(f) Mass spectrometry: Matrix-assisted laser desorption/ionization time-of-flight mass spectrometry (MALDI-TOF-MS) may be used to compare DNA fragments by sensitive mass determination.

(g) Southern blotting: a labelled probe consisting of a fragment of the linkage region is hybridised to nylon membranes containing genomic DNA from patients and normal controls digested with different restriction enzymes. Large differences in the sizes of the restriction fragments hybridizing with the probe between patients and controls may indicate the presence of a restriction fragment length polymorphism.

The above-listed techniques may be employed to scan the linkage region in order to identify novel polymorphic variants.

A significant amount of information regarding known polymorphic variants is to be found in publicly accessible databases such as, for example, the human SNP database accessible via the Website of the Whitehead Institute, Cambridge, Mass., USA (www-genome.wi.mit.edu/SNP/human/index.html). Thus, the invention also contemplates scanning/searching of these sources to identify polymorphic variants within the linkage region which may be associated with dermal inflammation and immunity. The association of any known polymorphisms identified using this approach with dermal inflammation and immunity in general or with a particular condition, such as atopic dermatitis, may again be investigated using genetic techniques, for example a family-based association study.

In a second aspect the invention comprises use of a region of human chromosome 1q21 including polymorphic marker D1S498 for identifying at least one human gene, including mutated or polymorphic variants thereof, which is associated with disease conditions involving dermal inflammation or immunity.

The invention also comprises use of a region of human chromosome 17q25 including polymorphic marker D17S784 for identifying at least one human gene, including mutated or polymorphic variants thereof, which is associated with disease conditions involving dermal inflammation or immunity.

The invention still further comprises use of a region of human chromosome 20p including polymorphic marker D20S115 for identifying at least one human gene, including mutated or polymorphic variants thereof, which is associated with a disease condition involving dermal inflammation or immunity.

This aspect of the invention also encompasses use of the linkage regions defined by reference to markers flanking the marker at the peak of linkage (see above) and also to sub-regions thereof.

There are many techniques known in the art by which a gene or genes associated with disease conditions involving dermal inflammation and immunity may be identified in the linkage regions defined herein (see Ausubel, F. M. et al. Current Protocols in Molecular Biology, Vol. 1,1989; Green Inc. New York, at 2.10.3). A non-limiting list of preferred techniques is provided below:

(a) Exon trapping: this is an artificial RNA splicing assay. Commonly the technique is performed using an exon-trap cosmid vector containing an artificial minigene consisting of a segment of a viral genome (e.g. SV40) containing an origin of replication and a powerful promoter sequence, two splicing-competent exons separated by an intron which contains a multiple cloning site and a polyadenylation signal. DNA from the linkage region is cloned into the exon-trap vector and the recombinant DNA transferred into a mammalian host cell. Transcription from the viral promoter results in an RNA transcript which normally splices to include the two exons of the minigene. If the cloned fragment of the linkage region itself contains a functional exon it will be incorporated into the spliced transcript. Spliced transcripts including a fragment of the linkage region can be identified using reverse transcription-PCR. Thus, the exon trap procedure can be used to identify coding regions in cloned fragments of the linkage region. Fragments containing a coding region can may then be used as probes in the identification of the remainder of the gene using standard molecular biology techniques.

(b) cDNA selection or capture (also known as direct selection and cDNA selection): this method involves forming genomic DNA/cDNA heteroduplexes by hybridizing a fragment of the linkage region to a complex mixture of cDNAs, such as the inserts of all the cDNA clones in a cDNA library. Related sequences will hybridise and can be enriched in subsequent steps, for example using PCR.

(c) Hybridization to mRNA/cDNA: a genomic fragment of the linkage region may be hybridized to a Northern blot of mRNA from a panel of different cell lines or against appropriate cDNA libraries. A positive hybridization signal can indicate the presence of a gene within the fragment.

(d) CpG island identification: CpG or HTF islands are short (about 1 kb) hypomethylated GC-rich (>60%) sequences which are often found at the 5′ ends of genes. CpG islands often comprise restriction sites for several rare-cutter restriction enzymes. Clustering of rare-cutter restriction sites is indicative of a CpG island and therefore of a possible gene. CpG islands can be detected by hybridization of a DNA clone to Southern blots of genomic DNA digested with rare-cutter enzymes, or by island-rescue PCR.

(e) Zoo-blotting: hybridizing a fragment of the linkage region at reduced stringency against a Southern blot of genomic DNA samples from a variety of animal species. Detection of hybridization signals can suggest conserved sequences, indicating a possible gene.

In addition to the above-described techniques, the availability of the human genome sequence in publicly accessible databases means that it may be possible to search for coding sequences in the linkage regions using in silico techniques, such as, for example homology searching and exon prediction. Human chromosomes 1, 17 and 20 have been completely sequenced as part of the human genome sequencing project and sequences are publicly available, for chromosomes 1 and 20 from the Sanger Centre, Hinxton, Cambridge, UK (www.sanger.ac.uk) and for chromosome 17 from the Whitehead Institute, Cambridge, Mass., USA. Thus, it is to be understood that ‘use’ of a particular linkage region in the identification of a gene or genes associated with dermal inflammation or immunity also encompasses ‘in silico’ use of the said region.

To further delineate the linkage regions associated with susceptibility for conditions involving dermal inflammation and immunity and assist in the identification of susceptibility genes additional fine mapping may be performed using additional markers in the linkage region. Newly identified markers are regularly made available in various publicly available sources such as, for example, the Cooperative Human Linkage Center (CHLC) public database, the public database of the Utah Center for Genome Research and databases maintained by the institutions forming the human genome sequencing consortium. Polymorphisms in the linkage region may be identified by searching publicly available SNP databases, such as the human SNP database accessible via the Whitehead Institute, Cambridge, Mass., USA. In addition, any novel polymorphic variants identified via scanning of the linkage region may also be used as additional markers for fine mapping.

Fine mapping may be performed by typing the additional markers in affected families, identifying linkage disequilibrium or association and thereby assembling a linkage disequilibrium map of the region.

To assist in the identification of susceptibility genes for conditions involving dermal inflammation and immunity clones of human genomic DNA covering the linkage regions may be assembled.

In a third aspect, the invention provides a method of predicting an individual's likelihood of developing a disease condition involving dermal inflammation or immunity, the method comprising:

obtaining a sample of the individual's DNA,

determining the individual's genotype in a region of chromosome 1q21 including polymorphic marker D1S498, and

comparing the individual's genotype to genotypes of affected subjects.

The invention further provides a method of predicting an individual's likelihood of developing a disease condition involving dermal inflammation or immunity, the method comprising:

obtaining a sample of the individual's DNA,

determining the individual's genotype in a region of chromosome 17q25 including polymorphic marker D17S784, and

comparing the individual's genotype to genotypes of affected subjects.

The invention still further provides a method of predicting an individual's likelihood of developing a disease condition involving dermal inflammation or immunity, the method comprising:

obtaining a sample of the individual's DNA,

determining the individual's genotype in a region of chromosome 20p including polymorphic marker D20S115, and

comparing the individual's genotype to genotypes of affected subjects.

This aspect of the invention also encompasses methods based on use of the linkage regions defined by reference to markers flanking the marker at the peak of linkage (see above) and also to sub-regions thereof.

The methods according to this third aspect of the invention may be used to predict an individual's general susceptibility for developing disease conditions which involve dermal inflammation and immunity. In a preferred embodiment, the method of the invention may be used to determine an individual's likelihood of developing atopic dermatitis.

In general terms the methods of the invention rely on determining the genotype of an individual in one of the chromosomal regions identified by the present inventors as being associated with general susceptibility to diseases involving dermal inflammation and immunity and comparing the individual's genotype to genotypes of affected subjects. An ‘affected subject’ is defined herein as an individual who has been diagnosed with a disease condition involving dermal inflammation and immunity (e.g. atopic dermatitis) using clinically accepted diagnostic criteria (discussed below for atopic dermatitis).

Data on the genotype of affected individuals may be assembled by direct sequencing of the relevant region of the chromosome or may be obtained by scanning of the relevant region for the presence of polymorphic variants using any of the techniques described above. Preferably, genotype data is collected from a large number of affected subjects. This data may then be used for comparison with genotype data obtained from the test individual. The degree of correspondence in genotype between the test individual and affected subjects is then used as an indication of susceptibility.

The aspect of the invention also contemplates methods of predicting an individual's likelihood of developing a disease condition involving dermal inflammation or immunity which involve determining the genotype of the individual at specific polymorphic loci associated with susceptibility to disease conditions involving dermal inflammation or immunity.

The invention will be further understood with reference to the following experimental report:

### EXPERIMENTAL 1

(a) Family Data

One hundred and forty-eight families containing 679 individuals were recruited from the dermatology clinics at the Great Ormond Street Hospital for Children, through a child or children with active AD. The clinics are for tertiary referrals, so that probands were anticipated to have AD at the severe end of the spectrum. The families contained 213 sibling pairs.

A physician-administered questionnaire was completed for each individual. The questionnaire included the modified Hanifin and Rajka diagnostic criteria for atopic dermatitis defined by the UK Working Party (Williams, H. C. et al., 1994a and 1994b) and questions based on the American Thoracic Society's questionnaire for respiratory disease (Mill, M. R. et al., 1995). Each family was examined for evidence of atopic dermatitis by a physician. The severity of AD was assessed using the scoring system of Rajka and Langeland, which categorizes patients into mild (3.0-4.0), moderate (4.5-7.5) or severe (8.0-9.0) disease on the basis of surface area involvement, continuity of disease and nocturnal pruritus (Rajka and Langeland, 1989). Asthma was defined on the basis of the questionnaire and a previous physician diagnosis as described previously (Cox H. E. et al., 1998). The total serum IgE concentration was measured by a fluorescent enzyme immuno-assay (Pharmacia CAP system, Pharmacia, Uppsala, Sweden).

The mean age of all the children in the study was 6.9 years (Standard Deviation ±3.9). One hundred and 72 were male and 166 female. Two hundred and 54 children had AD, 153 had asthma and 139 had both. The mean age of the children with AD was 6.9+4.4 years: 124 were male and 130 female. The geometric mean age of onset of disease was 1.5 years, and 90% of the children had an age of onset less than 2 years. The mean severity score was 6.2±1.9. Fifty-one point five % of children had moderate disease and 28.6% had severe disease. The serum IgE concentration was higher in children with AD and asthma together (geometric mean 880 IU/l; 95% Confidence Interval 637-1230 IU/l) than in children with asthma alone (mean 91; 95% CI 23-361 IU/l) or with AD alone (mean 171; 95% CI 106-277 IU/l).

(b) Genotyping of Family Members

Three hundred and 85 fluorescently labelled microsatellite markers from the ABI LMS2 panel were typed as previously described (Daniels, S. E. et al., 1996), giving a total of 210,161 genotypes. The average marker spacing was ˜8.9 cM and the average information content across the genome was >65%. Marker order was taken from the Marshfield map (Broman K. W. et al., 1998) and the Whitehead physical map (http://www-genome.wi.mit.edu/). Mendelian errors were removed before linkage analysis.

(c) Statistical Analysis

Four phenotypic models were tested for linkage against the marker set by non-parametric sib-pair methods. These were ADao (affected subjects only), ADau (affected and unaffected subjects given equal weighting), asthmaau (affected and unaffected subjects given equal weighting), and the total serum IgE analysed as a quantitative trait. There were insufficient subjects with asthma to analyse asthma by affected sib pairs only.

The probability of all possible inheritance vectors was calculated at each marker, and at four positions between markers, using the Lander-Green algorithm (Lander, E. S. and Green, P., 1987). For each family i a non-parametric linkage statistic was defined as Zᵢ=(Sᵢ−μᵢ)/σᵢ, where Sᵢ=E(S|marker data for family i), μᵢ=E(S|Hₒ) and σᵢ=Var(S|Hₒ). For each inheritance vector, the linkage score was defined as

$$S = {\sum }_{f} {( {\sum }_{j ∈ carriers\_of \_f} {y}_{j} )}^{2}$$

(f denotes the founder alleles in the pedigree). Phenotypes were scored as 1 (affected) and 0 (unaffected) for affecteds only analyses. For analyses incorporating affected and unaffected individuals, phenotypes for unaffected individuals were scored as −1. Residuals after adjustment for age and sex were used for the total serum IgE concentration, as described previously (Dizier, M. H. et al., 1995). The likelihood of the data and a one-parameter test for linkage based on the Zᵢ were constructed as described by Kong and Cox, 1997. All families were assigned equal weights.

To calculate genome-wide significance levels, 10,000 simulated genome-scans were generated under the null hypothesis of no linkage, but retaining the pattern of missing data and marker spacing observed in the original genome scan. The four phenotypes from the original screen were analysed in each simulation. The probability P_{c} of observing linkage to one or more of the phenotypes was calculated for each chromosome as P_{c}=(number of replicates with a χ² threshold on chromosome c)/10,000. The probability of observing n linkage peaks is then

$${\sum }_{x} {\prod }_{c} {{p}_{c}^{{x}_{c}} ( 1 - {p}_{c} )}^{1 - {x}_{c}} ,$$

where x is a vector of length 22 (the number of chromosomes) with n digits set to 1 and 22−n digits set to 0. The probability of observing linkage within ±25 cM of the previously reported psoriasis loci was calculated in an analogous manner.

(d) Results

Evidence for linkage to AD was identified on chromosome 1q21 (p=0.0005) and chromosome 17q25 (p=0.0004)(Table 1). Asthma was linked to chromosome 20p (p=0.0005). Linkage of this locus to children with both AD and asthma was not greatly different than to asthma alone (x²=10.9, p=0.0005), suggesting that the combination of AD and asthma may correspond to a genetic sub-type of both diseases. The total serum IgE concentration was linked to chromosome 16q (p=0.0004).

Monte-Carlo simulations were then carried out to estimate the true genome-wide significance levels for the supposed linkages. 10,000 simulated genome-scans were generated using the same four phenotypes and the pattern of data and marker spacing observed in the original genome scan. The results indicated that 0.86 linkages would be expected by chance, and that the probability that all four linkages were spurious was less than 1% (Table 2).

The putative chromosome 1q21, 17q25 and 20p loci identified in the present study are closely coincident with regions known to contain psoriasis susceptibility genes (Capon et al., 1999; Tomfohrde et al., 1994; Trembath et al., 1997)(Table 3). The chromosome 17q25 and 20p loci have not been characterised in any detail, but a cluster of genes in the chromosome 1q21 locus are known to be involved in the regulation of epidermal differentiation (Wjst et al., 1999).

Six major psoriasis loci are recognised (Capon et al., 1999; Tomfohrde et al., 1994; Trembath et al., 1997; Enlund et al., 1999a; Matthews et al., 1996; Nair et al., 1997; Enlund et al., 1999b). The significance of coincidence of the putative AD loci with these 6 regions was therefore estimated by further Monte Carlo simulations.

These results showed that the probability of three loci from the present screen coinciding within ±25 cM of known psoriasis loci was 2.8×10−5 (Table 4). The ±25 cM interval for co-localisation is conservative, as the three peaks of linkage from the present screen actually share markers with peaks for psoriasis loci (D1S498, D17S784 and D20S186) (Table 1 and Table 3).

TABLE 1
Results of linkage analysis from genome screen
Linkages with p < 0.001 are shown, together with
flanking markers with p < 0.05.

| | | ADₐₙ | | ADₐₙ | | Asthmaₐₙ | | IgE | |
|---|---|---|---|---|---|---|---|---|---|
| | Location | | | | | | | | |
| Marker | * | χ²(LR)^{z,801} | p** | χ²(LR) | p | χ^{2(LR)} | p | χ²(LR) | p |
| D1S252 | 155.1 | 4.74 | 0.015 | 7.54 | 0.003 | — | — | 3.45 | 0.03 |
| D1S498 | 160.7 | 4.00 | 0.02 | 10.95 | 0.0005 | — | — | 3.04 | 0.04 |
| D1S484 | 173.9 | — | — | 5.34 | 0.01 | — | — | — | — |
| D16S520 | 123.3 | — | — | — | — | — | — | 10.25 | 0.0007 |
| D17S784 | 117.7 | 11.04 | 0.0004 | 5.38 | 0.01 | — | 13 | — | — |
| D17S928 | 128.7 | 8.23 | 0.002 | 4.78 | 0.015 | — | — | — | — |
| D20S889 | 11.0 | — | — | — | — | 3.86 | 0.02 | — | — |
| D20S115 | 20.9 | — | — | — | — | 10.63 | 0.0005 | — | — |
| D20S186 | 33.2 | — | — | — | — | 6.67 | 0.01 | — | — |

* Position in cM from top of chromosome linkage group [symbol]
Likelihood ratio χ²
**Single marker significance, unadjusted for genome wide scan

TABLE 2
Results of simulation for genome-wide significance
levels
The results of the study are compared with 10,000
simulated genome screens.

| | Probability all |
|---|---|
| Observed Number | linkages occurring by |
| of Loci | chance |
| 1 | 3.73e−01 |
| 2 | 1.59e−01 |
| 3 | 4.29e−02 |
| 4 | 8.17e−03 |
| 5 | 1.17e−03 |

TABLE 3
Previously observed linkages to psoriasis

| | | Marker at peak of | |
|---|---|---|---|
| Chromosome | Study | linkage | Location cM |
| 1q21 | 1114 | D1S498 | 160.7 |
| 3q21 | 15 | D3S1269 | 142.2 |
| 4q | 16 | D4S1535 | 198.5 |
| 6p (MHC) | 1318 | D6S273 | 44.9 |
| 17q25 | 121718 | D17S784 | 117.7 |
| 20p | 1317 | D20S186 | 33.2 |

TABLE 4
Results of simulations for significance of coincidence
of AD and psoriasis loci
The results of 10,000 simulated genome screens show
how many coincident linkages are expected within 25 cM
in either direction of the linkages in Table 3.

| | Probability all |
|---|---|
| Observed Number | coincident linkages |
| of coincident Loci | occurring by chance |
| 1 | 6.43e−02 |
| 2 | 1.83e−03 |
| 3 | 2.77e−05 |
| 4 | 2.34e−07 |
| 5 | 1.04e−09 |

### REFERENCES

1. Juhlin, L., Johansson, G., Bennich, H., Hogman, C. & Thyresson, N. Immunoglobulin E in dermatoses. Levels in atopic dermatitis and urticaria. Arch Dermatol 100, p12-6 (1969).

2. Cox, H. E. et al. Association of atopic dermatitis to the beta subunit of the high affinity immunoglobulin E receptor. Br J Dermatol 138, 182-7 (1998).

3. Bos, J. Immunology of Atopic Dermatitis (eds. Harper, J., Oranje, A. & Prose, N.) (Blackwell Science, Oxford, 2000).

4. Larsen, F. S., Holm, N. V. & Henningsen, K. Atopic dermatitis. A genetic-epidemiologic study in a population-based twin sample. J Am Acad Dermatol 15, 487-94 (1986).

5. Schultz Larsen, F. Atopic dermatitis: a genetic-epidemiologic study in a population-based twin sample. J Am Acad Dermatol 28, 719-23 (1993).

6. Daniels, S. E. et al. A genome-wide search for quantitative trait loci underlying asthma. Nature 383, 247-50 (1996).

7. Ober, C. et al. Genome-wide search for asthma susceptibility loci in a founder population. The Collaborative Study on the Genetics of Asthma. Hum Mol Genet 7, 1393-8 (1998).

8. Wjst, M. et al. A genome-wide search for linkage to asthma. German Asthma Genetics Group. Genomics 58, 1-8 (1999).

9. Becker, K. et al. Clustering of non-major histocompatibility complex susceptibility candidate loci in human autoimmune diseases. Proc Natl Acad Sci USA 95, p9979-84 (1998).

10. Cookson, W. The alliance of genes and environment in asthma and allergy. Nature 402, B5-11 (1999).

11. Capon, F. et al. Searching for psoriasis susceptibility genes in Italy: genome scan and evidence for a new locus on chromosome 1. J Invest Dermatol 112, p32-5 (1999).

12. Tomfohrde, J. et al. Gene for familial psoriasis susceptibility mapped to the distal end of human chromosome 17q. Science 264, p1141-5 (1994).

13. Trembath, R. et al. Identification of a major susceptibility locus on chromosome 6p and evidence for further disease loci revealed by a two stage genome-wide search in psoriasis. Hum Mol Genet 6, p813-20 (1997).

14. Hardas, B. et al. Assignment of psoriasin to human chromosomal band 1q21: coordinate overexpression of clustered genes in psoriasis. J Invest Dermatol 106, p753-8 (1996).

15. Enlund, F. et al. Psoriasis susceptibility locus in chromosome region 3q21 identified in patients from southwest Sweden. Eur J Hum Genet 7, p783-90 (1999).

16. Matthews, D. et al. Evidence that a locus for familial psoriasis maps to chromosome 4q. Nat Genet 14, 231-3 (1996).

17. Nair, R. et al. Evidence for two psoriasis susceptibility loci (HLA and 17q) and two novel candidate regions (16q and 20p) by genome-wide scan. Hum Mol Genet 6, p1349-56 (1997).

18. Enlund, F. et al. Analysis of three suggested psoriasis susceptibility loci in a large Swedish set of families: confirmation of linkage to chromosome 6p (HLA region), and to 17q, but not to 4q. Hum Hered 49, p2-8 (1999).

19. Camp, R. in Textbook of Dermatology (eds. Champion, R., Burton, J., Burns, D. & Breathnach, S.) 1589-1649 (Blackwell Science, Oxford, 1998).

20. Farber, E., Nall, M. & Watson, W. Natural history of psoriasis in 61 twin pairs. Arch Dermatol 109, p207-11 (1974).

21. Brandrup, F., Hauge, M., Henningsen, K. & Eriksen, B. Psoriasis in an unselected series of twins. Arch Dermatol 114, p874-8 (1978).

22. Bos, J. D. The skin as an organ of immunity. Clin Exp Immunol 107 Suppl 1, 3-5 (1997).

23. Bos, J. D. et al. The skin immune system (SIS): distribution and immunophenotype of lymphocyte subpopulations in normal human skin. J Invest Dermatol 88, 569-73 (1987).

24. Folster-Holst, R. et al. Linkage between atopy and the IgE high-affinity receptor gene at 11q13 in atopic dermatitis families. Hum Genet 102, p236-9 (1998).

25. Williams, H. C., Burney, P. G., Pembroke, A. C. & Hay, R. J. The U.K. Working Party's Diagnostic Criteria for Atopic Dermatitis. III. Independent hospital validation. Br J Dermatol 131, 406-16 (1994).

26. Williams, H. C. et al. The U.K. Working Party's Diagnostic Criteria for Atopic Dermatitis. I. Derivation of a minimum set of discriminators for atopic dermatitis. Br J Dermatol 131, 383-96 (1994).

27. Cookson, W. O. C. M. & Hopkin, J. M. Dominant inheritance of atopic immonoglobulin-E responsiveness. Lancet 1, 86-88 (1988).

28. Rajka, G. & Langeland, T. Grading of the severity of atopic dermatitis. Acta Dermatology Venereology 144, 13-14 (1989).

29. Broman, K. W., Murray, J. C., Sheffield, V. C., White, R. L. & Weber, J. L. Comprehensive human genetic maps: individual and sex-specific variation in recombination. Am J Hum Genet 63, 861-9 (1998).

30. Lander, E. S. & Green, P. Construction of multilocus genetic linkage maps in humans. Proc Natl Acad Sci USA 84, 2363-7 (1987).

31. Dizier, M. H. et al. Detection of a recessive major gene for high IgE levels acting independently of specific response to allergens. Genet Epidemiol 12, 93-105 (1995).

32. Kong, A. & Cox, N.J. Allele-sharing models: LOD scores and accurate linkage tests. Am J Hum Genet 61, 1179-88 (1997).

1. Use of a region of human chromosome 1q21 including polymorphic marker D1S498 for identifying a polymorphic allele which is associated with susceptibility to disease conditions involving dermal inflammation or immunity.

2. The use as claimed in claim 1 wherein the region of human chromosome 1q21 including polymorphic marker D1S498 is the region disposed between polymorphic markers D1S2726 and D1S2878 or a sub-region thereof.

3. Use of a region of human chromosome 17q25 including polymorphic marker D17S784 for identifying a polymorphic allele which is associated with susceptibility to disease conditions involving dermal inflammation or immunity.

4. The use as claimed in claim 3 wherein the region of human chromosome 17q25 including polymorphic marker D17S784 is the region disposed between polymorphic markers D17S785 and the telomere or a sub-region thereof.

5. Use of a region of human chromosome 20p including polymorphic marker D20S115 for identifying a polymorphic allele which is associated with susceptibility to disease conditions involving dermal inflammation or immunity.

6. The use as claimed in claim 5 wherein the region of human chromosome 20p including polymorphic marker D20S115 is the region disposed between polymorphic markers D20S117 and D20S112 or a sub-region thereof.

7. The use according to any one of claims 1 to 6 for identifying a polymorphic allele which is associated with susceptibility to atopic dermatitis.

8. Use of a region of human chromosome 1q21 including polymorphic marker D1S498 for identifying at least one human gene, including mutated or polymorphic variants thereof, which is associated with disease conditions involving dermal inflammation or immunity.

9. The use as claimed in claim 8 wherein the region of human chromosome 1q21 including polymorphic marker D1S498 is the region disposed between polymorphic markers D1S2726 and D1S2878 or a sub-region thereof.

10. Use of a region of human chromosome 17q25 including polymorphic marker D17S784 for identifying at least one human gene, including mutated or polymorphic variants thereof, which is associated with disease conditions involving dermal inflammation or immunity.

11. The use as claimed in claim 10 wherein the region of human chromosome 17q25 including polymorphic marker D17S784 is the region disposed between polymorphic markers D17S785 and the telomere or a sub-region thereof.

12. Use of a region of human chromosome 20p including polymorphic marker D20S115 for identifying at least one human gene, including mutated or polymorphic variants thereof, which is associated with a disease condition involving dermal inflammation or immunity.

13. The use as claimed in claim 12 wherein the region of human chromosome 20p including polymorphic marker D20S115 is the region disposed between polymorphic markers D20S117 and D20S112 or a sub-region thereof.

14. The use according to any one of claims 8 to 13 for identifying at least one human gene, including mutated or polymorphic variants thereof, which is associated with atopic dermatitis.

15. A method of predicting an individual's likelihood of developing a disease condition involving dermal inflammation or immunity, the method comprising:
obtaining a sample of the individual's DNA,
determining the individual's genotype in a region of chromosome 1q21 including polymorphic marker D1S498, and
comparing the individual's genotype to genotypes of affected individuals.

16. A method as claimed in claim 15 wherein the region of human chromosome 1q21 including polymorphic marker D1S498 is the region disposed between polymorphic markers D1S2726 and D1S2878 or or a sub-region thereof.

17. A method of predicting an individual's likelihood of developing a disease condition involving dermal inflammation or immunity, the method comprising:
obtaining a sample of the individual's DNA,
determining the individual's genotype in a region of chromosome 17q25 including polymorphic marker D17S784, and
comparing the individual's genotype to genotypes of affected individuals.

18. A method as claimed in claim 17 wherein the region of human chromosome 17q25 including polymorphic marker D17S784 is the region disposed between polymorphic markers D17S785 and the telomere or a sub-region thereof.

19. A method of predicting an individual's likelihood of developing a disease condition involving dermal inflammation or immunity, the method comprising:
obtaining a sample of the individual's DNA,
determining the individual's genotype in a region of chromosome 20p including polymorphic marker D20S115, and
comparing the individual's genotype to genotypes of affected individuals.

20. A method as claimed in claim 19 wherein the region of human chromosome 20p including polymorphic marker D20S115 is the region disposed between polymorphic markers D20S117 and D20S112 or a sub-region thereof.

21. A method according to any one of claims 15 to 20 wherein the disease condition is atopic dermatitis.
